(19) United States
(12) Patent Application Publication
(10) Pub. No.: US 2017/0156895 A1
(43) Pub. Date: Jun. 8, 2017

(54) MOVEMENT ASSISTANCE SYSTEM AND METHOD THEREOF

(71) Applicant: NATIONAL CHIAO TUNG UNIVERSITY, Hsinchu City (TW)

(72) Inventors: Kai-Tai SONG, New Taipei City (TW); Yu-Lin CHIEN, Taichung City (TW)

(21) Appl. No.: 14/957,620

(22) Filed: Dec. 3, 2015

(51) Int. Cl. A61F 2/72; A61H 3/00; A63B 26/00; A61F 2/60; A61F 2/64

(52) U.S. Cl. CPC A61F 2/72; A61F 2/60; A61F 2/64; A63B 26/003; A61H 3/00

(57) ABSTRACT

A movement assistance method includes following steps: detecting a center of pressure; generating a gravity scale according to the center of pressure; measuring a joint angle and estimating a user external torque and a motor torque; generating a compensated assistive torque according to the user external torque, the joint angle, the motor torque and the gravity scale.

### BACKGROUND

Field of Invention

The present disclosure relates to a movement assistance system and method thereof. More particularly, the present disclosure relates to movement assistance system and method thereof that can be applied for a lower limb exoskeleton.

Description of Related Art

In recent years, the movement assistance system is increasing popular. The movement assistance system, such as lower limb exoskeleton, can be used for helping people to stand, walk or move with less strength while people wear it. Further, the lower limb exoskeleton also can be applied to support user to rehabilitate and to help for spiral injured patients. The lower limb exoskeleton works to enhance the user's strength of lower limb by a control system and mechanism.

It is important that the lower limb exoskeleton has to provide a safety and comfort mechanism to help user walk easily and keep their balance. Therefore, how to develop a lower limb exoskeleton to support elderly people to walk in balance becomes a problem to be solved in art.

### SUMMARY

One aspect of the present disclosure is related to a movement assistance system. In accordance with one embodiment of the present disclosure, the movement assistance system includes: a plurality of pressure sensors and a processing circuit. The pressure sensors are used for detecting a center of pressure (COP). The processing circuit includes: a user balance gravity scale generation module, an external torque estimation module and an assistive torque generation module. The user balance gravity scale generation module is used for generating a gravity scale according to the center of pressure. The external torque estimation module is used for measuring a joint angle and estimating a user external torque and a motor torque. The assistive torque generation module is used for generating a compensated assistive torque according to the user external torque, the joint angle, the motor torque and the gravity scale.

Another aspect of the present disclosure is related to a movement assistance method. The movement assistance method includes: detecting a center of pressure; generating a gravity scale according to the center of pressure; measuring a joint angle and estimating a user external torque and a motor torque; generating a compensated assistive torque according to the user external torque, the joint angle, the motor torque and the gravity scale.

These and other features, aspects, and advantages of the present disclosure will become better understood with reference to the following description and appended claims. Through utilizing one embodiment described above, the disclosure obtains the center of pressure of the user and an estimate user's balance state by measuring user external torque and motor torque so that it can improve user's balance during walking. The estimation of COP is used it for generating the correction torque. This correction torque will help user to keep their balance when they tend to lose their balance.

### BRIEF DESCRIPTION OF THE DRAWINGS

The disclosure can be more fully understood by reading the following detailed description of the embodiment, with reference made to the accompanying drawings as follows:

FIG. 1 is a schematic diagram of a movement assistance system according to one embodiment of the present disclosure.

FIG. 2 is a block diagram of a movement assistance system according to one embodiment of the present disclosure.

FIG. 3 is a flowchart of a movement assistance method according to one embodiment of the present disclosure.

FIG. 4 is a schematic diagram of pressure sensor placement for center of pressure measurement according to one embodiment of the present disclosure.

### DETAILED DESCRIPTION

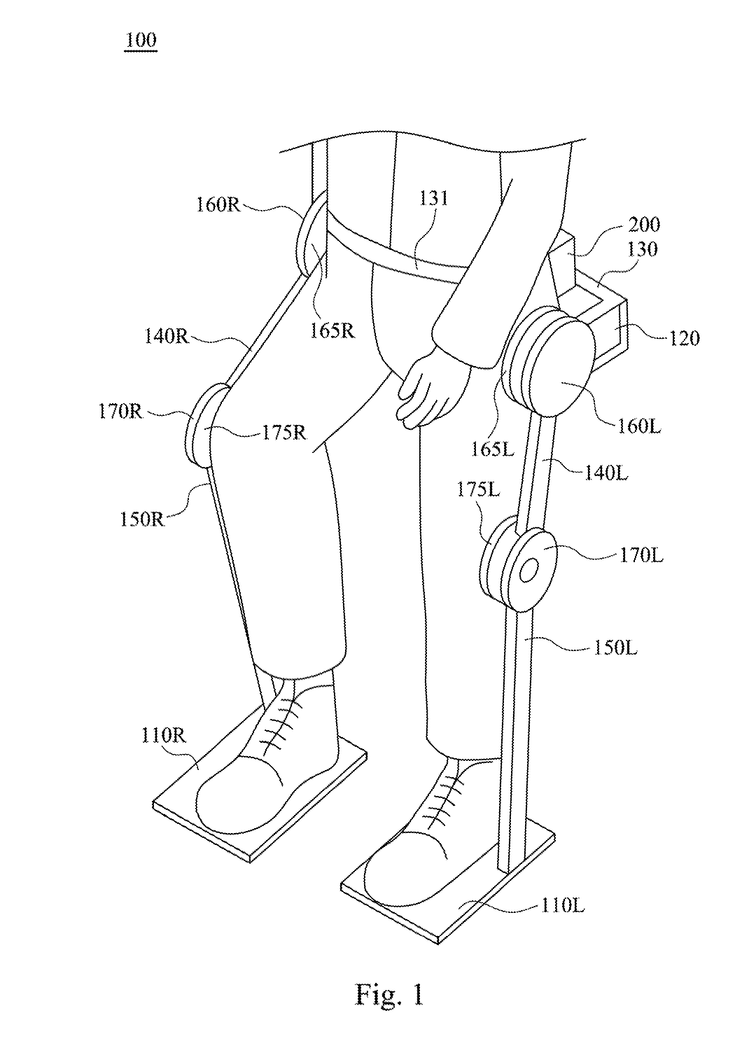

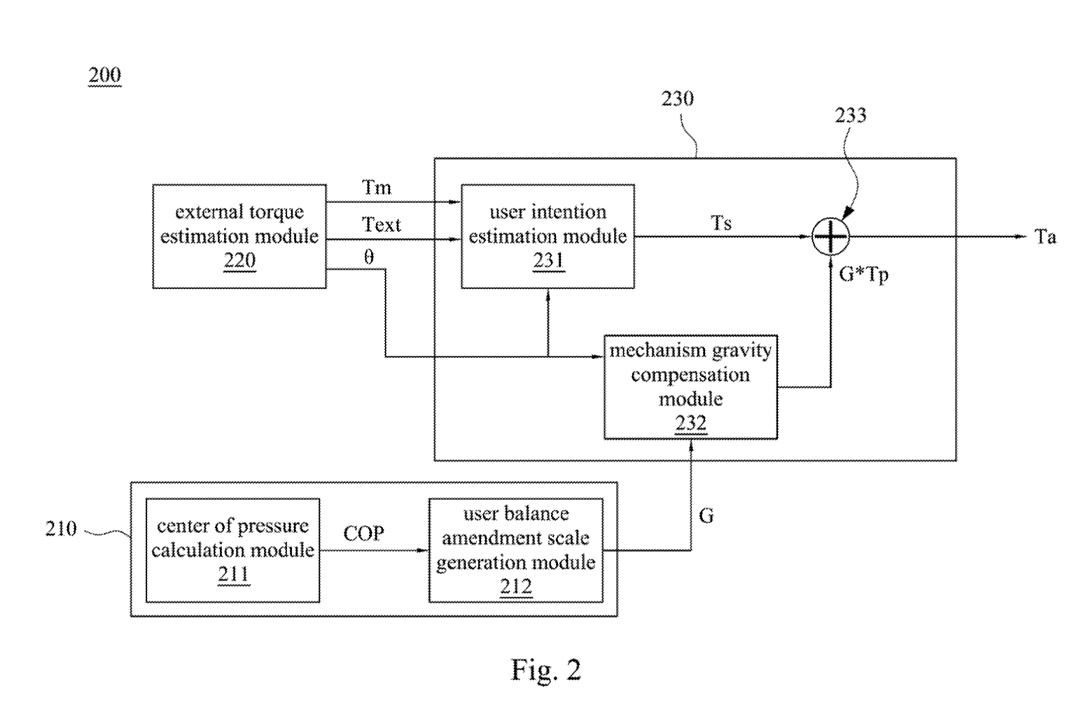

Reference is made to FIGS. 1-2. FIG. 1 is a schematic diagram of a movement assistance system 100 according to one embodiment of the present disclosure. FIG. 2 is a block diagram of a movement assistance system according to one embodiment of the present disclosure.

As shown in FIG. 1, the movement assistance system 100 includes a plurality of pressure sensors (not shown) and a processing circuit 200. To be more specified, as shown in FIG. 2, the processing circuit 200 includes: a user balance gravity scale generation module 210, an external torque estimation module 220 and an assistive torque generation module 230. The user balance gravity scale generation module 210 is used for generating a gravity scale G according to the center of pressure (COP). The external torque estimation module 220 is used for measuring a joint angle θ and estimating a user external torque Text and a motor torque Tm. The assistive torque generation module 230 is used for generating a compensated assistive torque Ta according to the user external torque Text, the joint angle θ, the motor torque Tm and the gravity scale G.

In one embodiment, the pressure sensors were set under both feet to detect the user's COP. For example, there are four pressure sensors placed on the lowest step broad 110L, and also four pressure sensors placed on the lowest step broad 110R. The lowest step broads 110L and 110R electrically coupled to the processing circuit 200. The pressure sensors are used for detecting a COP.

In one embodiment, the user balance gravity scale generation module 210, an external torque estimation module 220 and an assistive torque generation module 230 can be implemented respectively or along with each other, and these modules can be implemented by a micro controller, a microprocessor, a digital signal processor, an application specific integrated circuit (ASIC), a firmware, a program, or a logical circuitry.

Based on above, the movement assistance system 100 uses pressure sensors that are set under both of user's feet. This movement assistance system 100 is used for obtaining the COP of the user and an estimate user's balance state so that it can improve user's balance during walking. The estimation of the COP is used for generating the correction torque. This correction torque will help user to keep their balance when: they tend to lose their balance. The technical feature related to the correction torque is further discussed later.

In one embodiment, as shown in FIG. 1, the movement assistance system 100 further includes a battery 120 for supplying power, a waist part of support structure 130 with a buckle fastener 131 for supporting a waist of a user, the upper part of support structures 140L, 140R separately for supporting thighs of the user, the lower part of support structures 150L, 150R separately for supporting calves of the user, the hip joints 165L, 165R for mechanically connecting with the waist part of support structure 130 and separately connecting with the upper part of support structures 140L, 140R, the knee joints 175L, 175R for mechanically and separately connecting with the upper part of support structures 140L, 140R. And, the knee joints 175L, 175R also mechanically and separately connecting with the lower part of support structure 150L, 150R. The knee motors 170L, 170R are used separately for controlling the knee joint 175L, 175R. And, the hip motors 160L, 160R are used separately for controlling the hip joint 165L, 165R. Wherein, the battery 120, the knee motors 170R, 170L, and the hip motors 160R, 160L are electronically coupled to the processing circuit 200. As such, processing circuit 200 can transmit a control signal according to the compensated assistive torque for controlling the knee motor 170L, the knee motor 170R, the hip motor 160L and/or hip motor 160R, separately.

In one embodiment, the length of the lower part of support structures 150L, 150R and the length of the upper part of support structures 140L, 140R can be adjusted longer or shorter according to the user's thighs and calves.

In one embodiment, the movement assistance system 100 can be implemented as only including one side mechanism for user to wear. For instance, user only wears the left part (e.g. processing circuit 200, battery 120, hip motor 160L, hip joint 165L, upper part of support structures 140L, knee motor 170L, knee joint 175L, lower part of support structure 150L, lowest step broad 110L) of the movement assistance system 100 for supporting the left lower limb.

In one embodiment, the processing circuit 200 is further coupled to a sensor control board. The sensor control board is used for controlling the pressure sensors and receiving the data detected from the pressure sensors. Therefore, the processing circuit 200 can obtain the COP data from the sensor control board. In one embodiment, the processing circuit 200 is further coupled to the motor controllers to separately control the operations of the knee motor 170L, the knee motor 170R, the hip motor 160L and/or the hip motor 160R. In one embodiment, these motor controllers are also coupled to the motor encoders. The motor encoders are used for separately monitoring the status of the knee motor 170L, knee motor 170R, hip motor 160L and hip motor 160R. The motor encoders can transmit the status data of each motor to the processing circuit 200.

Based on above, the movement assistance system 100 has the modular structures, the adjustable length of shank and the safe guard mechanism by calculating the compensated assistive torque Ta. It makes that the movement assistance system 100 works smoothly, comfortable, and prevent from the unexpected.

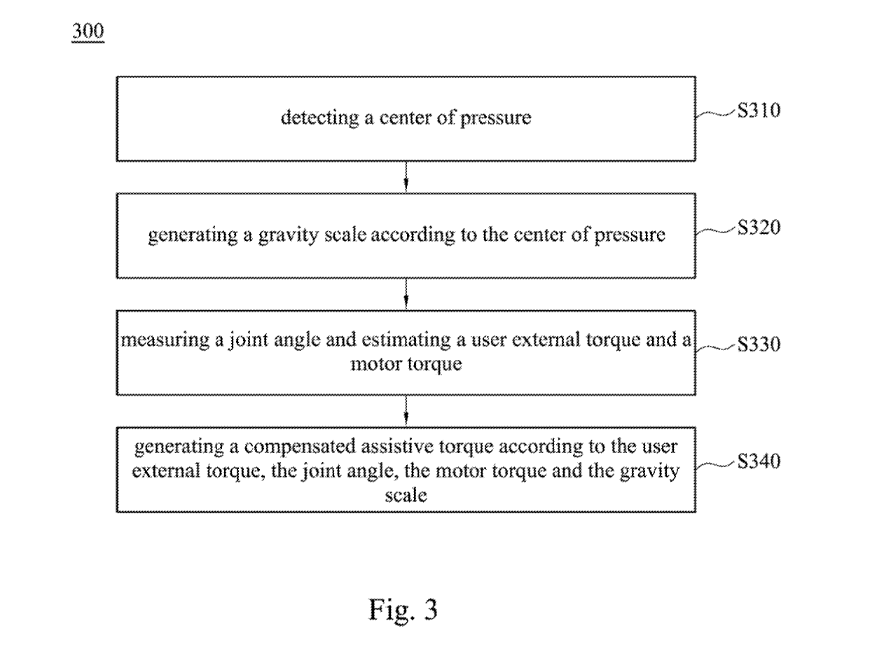

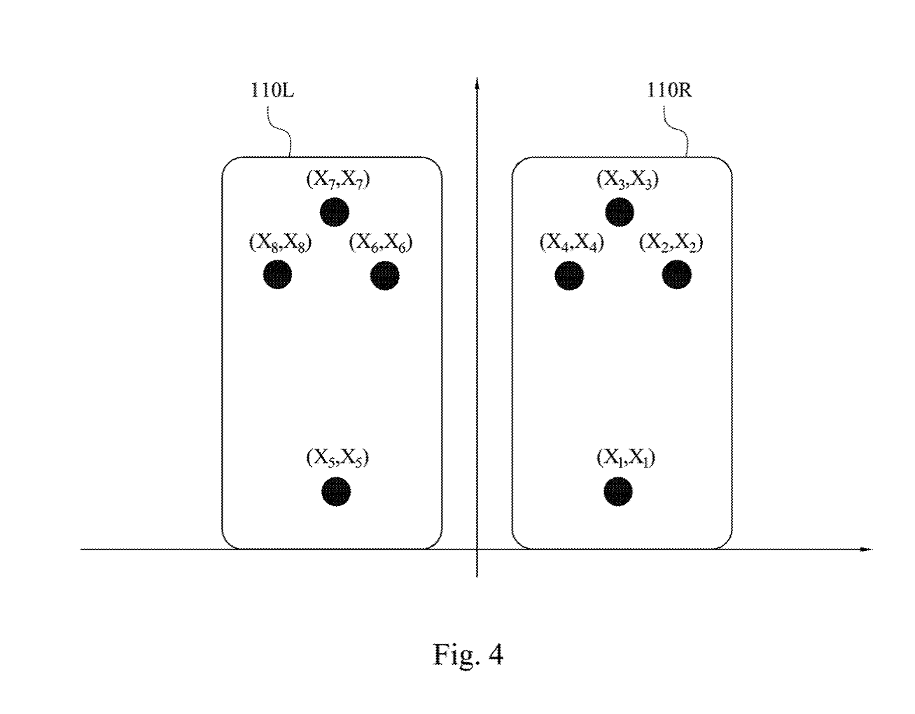

The movement assistance system 100 with the movement assistance method 300 is further described in the following paragraphs. Refer to FIGS. 3 and 4. FIG. 3 is a flowchart of a movement assistance method according to one embodiment of the present disclosure. FIG. 4 is a schematic diagram of pressure sensor placement for center of pressure measurement according to one embodiment of the present disclosure. To be convenient for explanation, the operation of the movement assistance system 100 will be described together with the movement assistance method 300.

In step S310, the pressure sensors are used for detecting a center of pressure (COP). In one embodiment, user's balance state can be analyzed according to the COP. For example, there are four pressure sensors placed on the lowest step broad 110L, and four pressure sensors placed on the lowest step broad 110R. And, the lowest step broad 110L, 110R are set under both of user's feet. As such, the COP is obtained by pressure sensors on each foot of lower limb exoskeleton, as shown in FIG. 4. The COP can be obtained by the pressure values corresponding to the coordinates of each pressure sensors.

For instance, each pressure sensors can detect the pressure values corresponding to its coordinates, such as (x₁, y₁), (x₂, y₂), (x₃, y₃), (x₄, y₄), (x₅, y₅), (x₆, y₆), (x₇, y₇) and (x₈, y₈).

In step S320, the user balance gravity scale generation module 210 is used for generating a gravity scale G according to the COP. In one embodiment, the user balance gravity scale generation: module 210 further includes a center of pressure calculation module 211 and a user balance amendment scale generation module 212. The center of pressure calculation module 211 receives the COP detected from the pressure sensors and transmits the COP to the user balance amendment scale generation module 212. The user balance amendment scale generation module 212 analyzes x-axis of the COP and y-axis of the COP. In this manner, the user balance amendment scale generation module 212 generating the gravity scale G according to the x-axis of the COP and y-axis of the COP. In one embodiment, the x-axis of the COP and y-axis of the COP can be corresponded to the known-patterns for generating the gravity scale G. It can be understood that a known fuzzy control system or any kinds of control system can be applied to generate the gravity scale G according to the x-axis of the COP and y-axis of the COP.

In step S330, the external torque estimation module 220 is used for measuring a joint angle θ and estimating a user external torque Text and a motor torque Tm. It is not limited to the operation sequence of the step S320 and the step S330.

In one embodiment, the joint angle θ is obtained according to the bending angle of the knee joint 175L, 175R or the hip joint 165L, 165R. In addition, the processing circuit 200 can receive the data, such as joint angle θ and motor torque Tm detected from the knee motor 170L, the knee motor 170R, the hip motor 160R and/or the hip motor 160L.

In one embodiment, the user external torque Text can be estimated by dynamic and balance state from the pressure sensor on feet. The dynamic model of the user external torque Text related to the knee motor 170L, the knee motor 170R, the hip motor 160R and/or the hip motor 160L that used in exoskeleton can be express in formula (1):

Text+Tm=Jα+Dω+Tg+f (1)

The left side of formula (1) is user external torque Text and motor torque Tm, and the right side is system responses. In formula (1), the notation J is motion inertia, notation α denotes angular acceleration, notation D is damping, notation ω is angular velocity, notation Tg is gravity effect on mechanism which is a function of hip and knee angle and f is friction force. According to the formula (1), the user external torque Text can be determined while the other parameters (e.g. motor torque Tm, motion. inertia J, angular acceleration α, damping D, angular velocity ω, gravity effect Tg and friction force f) are obtained.

The results of user external torque Text will use to estimate balance state when user wear the exoskeleton.

In step S340, the assistive torque generation module 230 is used for generating a compensated assistive torque Ta according to the user external torque Text, the joint angle θ, the motor torque Tm and the gravity scale G.

Throughout the assistive torque generation, the compensated assistive torque Ta were got from support torque when user wear the exoskeleton, the gravity compensation is developed to compensate the gravity effect which is depended on lower limb posture. This part also constitutes the correction torque to keep user balance while walking. The following embodiments provide the specified description which related to generate the compensated assistive torque Ta.

As a user wearing the lower limb exoskeleton, the system will provide the user with a support torque Ts at the appropriate timing during walking. In one embodiment, the assistive torque generation module 230 includes a user intention estimation module 231 for generating the support torque Ts according to the user external torque Text, the joint angle θ and the motor torque Tm. In one embodiment, the user intention estimation module 231 can generate the support torque Ts immediately when user intent to move.

Besides, the gravity effect on movement assistance system 100 would increase the load to user. As such, the gravity compensation torque Tp aims to compensate gravity effect and the value depends on lower limb posture. In one embodiment, the assistive torque generation module 230 includes a mechanism gravity compensation module 232 for receiving the gravity scale G and the joint angle θ, generating a gravity compensation torque Tp according to the joint angle θ. Wherein, the lower limb posture of user can affect the joint angle θ. For example the lower limb posture of user can affect the bending angle of the knee joint 175L, 175R or the hip joint 165L, 165R. And then, the mechanism gravity compensation module 232 multiplies the gravity scale G by the gravity compensation torque Tp to obtain a correction torque.

The correction torque will help user to keep their balance when they tend to lose their balance. The correction torque will send the positive torque when user climb and negative value when user walks downhill. In one embodiment, a fuzzy controller can be designed according to the estimation of COP to generate the gravity scale G, which affects the value and sign of correction torque. In one embodiment, the gravity scale G is a value from 0 to 2. The mechanism gravity compensation module 232 multiplies the gravity scale G by gravity compensation torque Tp to generate a correction torque.

In one embodiment, the assistive torque generation module 233 includes an assistive controller module 233 for receiving the support torque Ts and the correction torque, and generating the compensated assistive torque Ta according to the support torque Ts and the correction torque. In one embodiment, the assistive torque generation module 233 can add the support torque Ts to the correction torque for generating the compensated assistive torque Ta. However, the method for generating the compensated assistive torque Ta according to the support torque Ts and the correction torque is not limited thereto.

As mentioned above, the support torque Ts is based on user external torque Text. The gravity compensation torque Tp is designed based on the posture of lower limb exoskeleton. The correction torque is designed based on user balance state to help a user to keep balance when tend to lose balance or walking on a ramp. These torques are combined to provide the compensated assistive torque Ta of each join of the exoskeleton to assist a user during walking.

Through utilizing at least one embodiment described above, the movement assistance system 100 and movement assistance method 300 can increases user's strength and keeps his/her balance during walking. It can be used for walking assistance as well as for rehabilitation. Pressure sensors were set under both feet to detect the user's COP. The COP is included in the control loop to improve balance in walking. In the control design, the assistive torque from each motor is related to the support torque, the gravity compensation torque, and the correction torque. The support torque is generated based on user's external torque during walking. The gravity compensation torque is calculated by using posture data of the exoskeleton to compensate gravity effect of the mechanism. The correction torque is based on user balance state to allow a user to keep their balance when he/she tends to lost body balance or walks on a ramp. Thus, the movement assistance system 100 can work smoothly, comfortable, and prevent from the unexpected.

Although the present disclosure has been described in considerable detail with reference to certain embodiments thereof, other embodiments are possible. Therefore, the scope of the appended claims should not be limited to the description of the embodiments contained herein.

What is claimed is:

1. A movement assistance system, comprising:
a plurality of pressure sensors for detecting a center of pressure;
a processing circuit, comprising:
an user balance gravity scale generation module for generating a gravity scale according to the center of pressure;
an external torque estimation module for measuring a joint angle and estimating a user external torque and a motor torque; and
an assistive torque generation module for generating a compensated assistive torque according to the user external torque, the joint angle, the motor torque and the gravity scale.

2. The movement assistance system of claim 1, wherein the processing circuit transmits a control signal according to the compensated assistive torque for controlling a knee motor or a hip motor.

3. The movement assistance system of claim 1, further comprising:
a lowest step broad electrically coupled to the processing circuit;
wherein the pressure sensors are placed on the lowest step broad.

4. The movement assistance system of claim 1, wherein the user balance gravity scale generation module further comprising:
a center of pressure calculation module for receiving the center of pressure detected from the pressure sensors; and
a user balance amendment scale generation module for analyzing a x-axis of the center of pressure and a y-axis of the center of pressure; and generating the gravity scale according to the x-axis of the center of pressure and the y-axis of the center of pressure.

5. The movement assistance system of claim 1, wherein the assistive torque generation module further comprising:
a user intention estimation module for generating a support torque according to the user external torque, the joint angle and the motor torque.

6. The movement assistance system of claim 5, wherein the assistive torque generation module further comprising:
a mechanism gravity compensation module for receiving the gravity scale and the joint angle, generating a gravity compensation torque according to the joint angle, and then multiplying the gravity scale by the gravity compensation torque to obtain a correction torque.

7. The movement assistance system of claim 6, wherein the assistive torque generation module further comprising:
an assistive controller module for receiving the support torque and the correction torque, and generating the compensated assistive torque according to the support torque and the correction torque.

8. The movement assistance system of claim 1, further comprising:
a battery for supplying a power;
a waist part of support structure for supporting a waist of a user;
a upper part of support structure for supporting a thigh of the user;
a lower part of support structure for supporting a calf of the user;
a hip joint for mechanically connecting with the waist part of support structure and the upper part of support structure;
a knee joint for mechanically connecting with the upper part of support structure and the lower part of support structure
a knee motor for controlling the knee joint; and
a hip motor for controlling the hip joint;
wherein the battery, the knee motor, and the hip motor are electronically coupled to the processing circuit.

9. The movement assistance system of claim 8, wherein the joint angle is obtained according to the knee joint or the hip joint.

10. A movement assistance method, comprising:
detecting a center of pressure by a plurality of pressure sensors;
generating a gravity scale according to the center of pressure;
measuring a joint angle and estimating a user external torque and a motor torque; and
generating a compensated assistive torque according to the user external torque, the joint angle, the motor torque and the gravity scale.

11. The movement assistance method of claim 10, further comprising:
transmitting a control signal according to the compensated assistive torque for controlling a knee motor or a hip motor.

12. The movement assistance method of claim 10, wherein the pressure sensors are placed on a lowest step broad.

13. The movement assistance method of claim 10, further comprising:
receiving the center of pressure detected from the pressure sensors;
analyzing a x-axis of the center of pressure and a y-axis of the center of pressure; and
generating the gravity scale according to the x-axis of the center of pressure and the y-axis of the center of pressure.

14. The movement assistance method of claim 10, further comprising:
generating a support torque according to the user external torque, the joint angle and the motor torque.

15. The movement assistance method of claim 14, further comprising:
receiving the gravity scale and the joint angle;
generating a gravity compensation torque according to the joint angle; and then
multiplying the gravity scale by the gravity compensation torque to obtain a correction torque.

16. The movement assistance method of claim 15, further comprising:
receiving the support torque and the correction torque; and
generating the compensated assistive torque according to the support torque and the correction torque.

17. The movement assistance method of claim 10, further comprising:
a battery for supplying a power;
a waist part of support structure for supporting a waist of a user;
a upper part of support structure for supporting a thigh of the user;
a lower part of support structure for supporting a calf of the user;
a hip joint for mechanically connecting with the waist part of support structure and the upper part of support structure;
a knee joint for mechanically connecting with the upper part of support structure and the lower part of support structure
a knee motor for controlling the knee joint; and
a hip motor for controlling the hip joint;
wherein the battery, the knee motor, and the hip motor are electronically coupled to a processing circuit.

18. The movement assistance method of claim 17, wherein the joint angle is obtained according to the knee joint or the hip joint.
